(19) United States
(12) Patent Application Publication
(10) Pub. No.: US 2001/0016676 A1
(43) Pub. Date: Aug. 23, 2001

(54) Intra-aortic balloon pump condensation prevention system

(76) Inventors: Jonathan Williams, Montville, NJ (US); Robert Hoff, Cedar Grove, NJ (US); Yefim Kaushansky, Fairlawn, NJ (US)

Correspondence Address: Datascope Corp., 14 Philips Parkway, Montvale, NJ, 07645, US

(21) Appl. No.: 09/776,664

(22) Filed: Feb. 5, 2001

Related U.S. Application Data
(63) Continuation-in-part of application No. 09/296,063, filed on Apr. 21, 1999, now Pat. No. 6,210,319.

(51) Int. Cl. A61M 1/12

(52) U.S. Cl. 600/18

(57) ABSTRACT

An intra-aortic balloon pump condensation prevention system comprising a water vapor extraction element, such as a Nafion tube (made by Dupont Inc.), or a condensation element, such as a cold trap or cooled coiled tube, preferably connected in shunt with the intra-aortic balloon catheter.

### RELATED APPLICATIONS

This is a continuation-in-part application of parent application Ser. No. 09/296,063.

### BACKGROUND OF THE INVENTION

1. Field of the Invention

The invention relates to an intra-aortic balloon pump condensation prevention system. More particularly, the invention relates to an improved system for preventing water vapor from condensing in the shuttle gas of an intra-aortic balloon pump system.

2. Description of the Prior Art

Intra-aortic balloon (IAB) catheters are used in patients with left heart failure to augment the pumping action of the heart. The catheters, approximately one (1) meter long, have a balloon at the distal end. The catheter is typically inserted into the femoral artery and moved up the descending thoracic aorta until the distal tip of the balloon is positioned just below or distal to the left subclavian artery. The proximal end of the catheter remains outside of the patient's body. A passageway for inflating and deflating the balloon extends through the catheter and is connected at its proximal end to an external pump. The patient's central aortic pressure is used to time the balloon and the patient's ECG may be used to trigger balloon inflation in synchronous counterpulsation to the patient's heartbeat.

Typical dual lumen intra-aortic balloon catheters have an outer, flexible, plastic tube, which serves as the inflating and deflating gas passageway, and a central tube therethrough formed of plastic tubing, stainless steel tubing, or wire coil embedded in plastic tubing. A polyurethane compound is used to form the IAB's balloon. Helium gas is typically used as the “shuttle gas”, the gas used to inflate and deflate the IAB. To be effective, the IAB inflation and deflation must occur rapidly, e.g. in less than one eighth of a second.

During operation of the IAB, shuttle gas diffuses from the IAB into the patient's blood stream. Also, water vapor diffuses from the patient's blood stream into the IAB's shuttle gas.

The outer tube (membrane) of the IAB is thin (approximately 0.004 inches thick) and is generally made from a mixture of polyurethane and silicone. These materials are permeable to helium and water vapor, and thus they allow for the above mentioned diffusion.

To compensate for the foregoing loss of helium, intra-aortic balloon pumping (IABP) systems replace or add helium gas on a periodic basis. If the helium is not replaced on a periodic basis, then the balloon will not completely inflate, with a consequent reduction in therapy.

To compensate for the continuous diffusion of water vapor into the shuttle gas, some IABP systems incorporate a water vapor removal device which removes or lowers the concentration of water vapor in the shuttle gas.

If the concentration of the water vapor is not lowered, the water vapor will condense and appear as liquid water within the IABP's shuttle gas system. Over a sufficient period of time, the water can accumulate and impede the flow of shuttle gas within the IABP system.

Condensation can be prevented if the dew point temperature of the shuttle gas is kept lower than the IABP's ambient temperature. Prior art IABP systems prevent condensate accumulation by placing a cold trap in series with the IAB catheter. A cold trap is a metallic block with drillings for the passage of gas and for the collection of condensate (“sump”). A thermo-electric cooler (Peltier) is used to keep the cold trap colder than IABP's ambient temperature.

During IABP operation, the shuttle gas, preferably helium, flows through a drilling in the block. Due to the block's lower temperature, condensate forms within the block and flows (due to force of gravity) into a sump. Periodically, the sump is automatically emptied via a valve. Shuttle gas is lost during the emptying process. Consequently, the sump is emptied concurrently with the shuttle gas removal or replacement.

The prior art condensate prevention system has a number of drawbacks. First, emptying the sump causes a loss of shuttle gas. Therefore, the helium consumption of the prior art condensation prevention systems is higher. Furthermore, because the sump cannot be emptied without a loss of shuttle gas, emptying the sump requires a shutdown of the IABP system, which causes an interruption in therapy.

Second, the condensate trap (the block) is continuously warmed by the flow of high velocity shuttle gas. Consequently, the thermoelectric cooler consumes a large amount of electric power as it maintains the trap's correct operating temperature. High power consumption is undesirable because a larger IABP system power supply, battery charger, and battery is required to accommodate such consumption.

Third, drillings within the condensate trap, which are in-line with the catheter, increases the dead volume within the shuttle gas system. During balloon inflation and deflation the velocity of the shuttle gas is very high. To maintain high IAB inflate/deflate speeds it is important to prevent unnecessary pressure drops in the shuttle gas circuit. To accommodate this design requirement, drillings within the condensate trap are made quite large. Unfortunately, the large drillings increase the dead volume in the shuttle gas system, i.e. they add dead volume in series with the IAB gas path. Parasitic dead volume wastes IABP power and can reduce theraputic efficacy by increasing the pneumatic compliance of the shuttle gas system.

Fourth, the prior art systems require disassembly and replacement and/or sterilization of the condensate trap when contaminated by blood due to an IAB membrane perforation. Occasionally, arterial plaque deposits abrade the IAB's membrane and thereby cause perforations in the membrane. When this occurs, blood can enter the IAB and may contaminate components within the shuttle gas system.

A final drawback of the prior art condensate prevention system is that it is orientation sensitive, i.e. the condensate trap's sump relies on gravity. Orientation sensitivity of a system component limits the design flexibility of the entire IABP system.

While the prior art designs may be suitable for the particular purpose employed, or for general use, they would not be as suitable for the purposes of the present invention as disclosed hereafter.

### SUMMARY OF THE INVENTION

Accordingly, it is an object of one or more embodiments of the invention to produce an improved IABP condensation prevention system, using a water vapor extraction element, such as Nafion tubing (made by Dupont Inc.), as an agent to reduce the concentration of water vapor in the shuttle gas to levels which prevent condensation from forming, which overcomes one or more of the above mentioned drawbacks of the prior art condensation prevention systems.

It is another object of one or more embodiments of the invention to produce a condensate prevention system which consumes less helium.

It is yet another object of one or more embodiments of the invention to produce a condensate prevention system which does not require an interruption of therapy to empty a sump.

It is a further object of one or more embodiments of the invention to produce a condensate prevention system with a considerably lower power consumption.

It is a still further object of one or more embodiments of the invention to produce a condensate prevention system with minimum parasitic dead volume space and with a dead volume location, i.e. in shunt, which has a minimum effect on the inflate and deflate speed of the intra-aortic balloon.

It is still yet another object of one or more embodiments of the invention to produce a condensate prevention system which is inexpensive to repair and service if blood enters the IAB.

It is still a further object of one or more embodiments of the invention to produce a condensate prevention system which is not sensitive to its orientation, i.e. a system which does not rely on gravity.

The invention is an intra-aortic balloon pump condensation prevention system comprising a water vapor extraction element, such as a Nafion tube, or a condensation element, such as a cold trap or cooled coiled tube, in shunt with the intra-aortic balloon catheter.

In one embodiment, the water vapor extraction element may comprise a cold trap in shunt with the IAB catheter.

In another embodiment, the water vapor extraction element may comprise a tube, or other shaped confining device, made from a water or vapor permeable membrane. Said Nafion tube absorbs water vapor in the intra-aortic balloon catheter shuttle gas. The water is then extracted from the Nafion's tubing's exterior by means of a vacuum supply or a dry purge gas.

In yet another embodiment, the water vapor extraction element may comprise a cooled coiled section of plastic or metallic tubing. In this embodiment, condensate forms on the interior walls of the coiled section. Due to an arrangement of valves, the condensate in said coiled section of tubing is forced to migrate towards a sump. Water accumulated in the sump is later removed by the helium replacement process.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is a block diagram of an intra-aortic balloon pump system having a prior art condensation prevention system.

FIG. 2 is block diagram of an intra-aortic balloon pump system with an improved condensation prevention system with a water vapor extraction element in-line (in series) with the IAB catheter.

FIG. 3 is a block diagram of the preferred embodiment of the intra-aortic balloon pump system with an improved condensation prevention system in shunt with the IAB.

FIG. 3A is a block diagram of the intra-aortic balloon pump system illustrated in FIG. 3 with optional components illustrated.

FIG. 4 is a plain view of a safety disk component with its membrane in a low volume position.

FIG. 5 is a plain view of a safety disk component with its membrane in a high volume position.

FIG. 6 is a block diagram of an alternate embodiment of the intra-aortic balloon pump system with an improved condensation prevention system.

FIG. 7 is a block diagram of the intra-aortic balloon pump system of FIG. 6 with an alternate condensation prevention system including a block having a drilling.

### DETAILED DESCRIPTION OF THE PREFERRED EMBODIMENTS

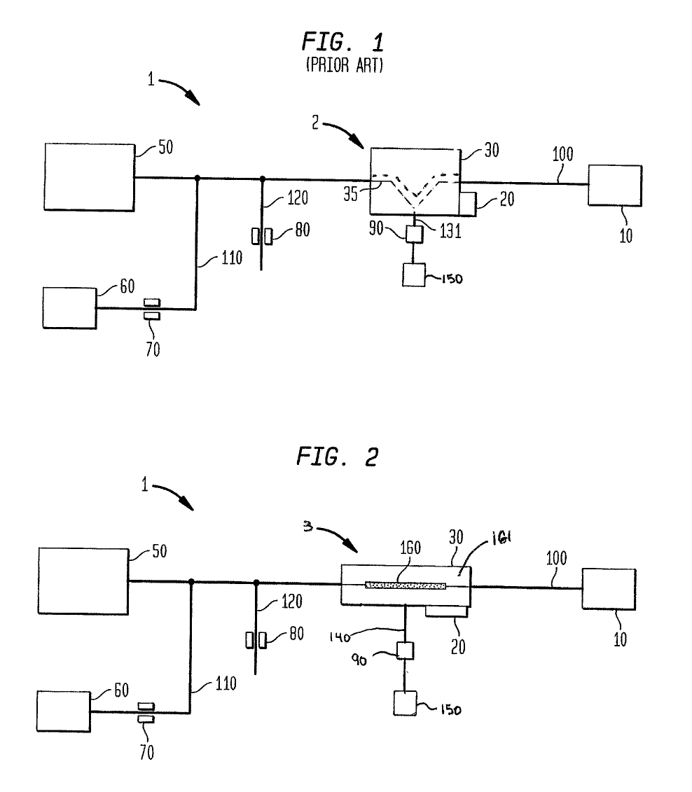

FIG. 1 illustrates a block diagram of an intra-aortic balloon pump (IABP) system, generally designated 1, in series with a prior art condensation prevention system, generally designated 2. IABP system 1 comprises an intra-aortic balloon (IAB) 10, a catheter 100, a pump 50, the condensation prevention system 2, a shuttle gas supply 60, a shuttle supply valve 70, a vent line 120, and a vent pinch valve 80. The design and manufacture of intra-aortic balloon catheters are generally known in the art, see for example U.S. Pat. No. 6,024,693, herein incorporated by reference in its entirety. Condensation prevention system 2 comprises a cooler 20, a cold trap 30, and a sump 90. Cold trap 30 and IAB 10 are connected by catheter 100. Cold trap 30 has a drilling 35 (shown in ghost lines) which communicates with catheter 100. The shuttle gas supply 60 is connected to the catheter 100 by a first line 110. The amount of shuttle gas supplied by the shuttle gas supply 60 is controlled by a shuttle gas supply valve 70. Shuttle gas is added to compensate for shuttle gas losses due to diffusion from the IAB to the patient, as discussed above. Helium is generally used for the shuttle gas. A second line 120 acts as a vent and is controlled by a vent pinch valve 80.

To initiate therapy, the shuttle gas system is purged of atmospheric gases via the action of the pump 50 and the vent components 120 and 80. Next, a precise amount of pure helium is admitted by opening the shuttle gas supply valve 70. During therapy, pump 50 moves shuttle gas back and forth to cause the inflation and deflation of IAB 10. As the shuttle gas shuttles back and forth in catheter 100, between the pump 50 and IAB 10, the shuttle gas passes through drilling 35 in the cold trap 30. The cold trap 30, a solid metallic block except for the drilling 35, is cooled by the cooler 20. As the shuttle gas is cooled, water vapor, which has diffused from the patient into the shuttle gas in IABP system 1, condenses in drilling 35 of cold trap 30 and trickles down into sump 90, which communicates with trap 30 by means of a third line 131. A vacuum supply 150 or vent line (not shown) is connected to sump 90 so as to allow for a periodic emptying of sump 90.

FIG. 2 illustrates a block diagram of an IABP system 1 with an improved condensation prevention system, generally designated 3. The improved IABP system 1 (FIG. 2) is physically similar to the prior art IABP system 1 (FIG. 1) except for the change in the condensation prevention system 3, specifically the incorporation of a water vapor extraction element 160 in line with catheter 100. Water vapor extraction element 160 is a tube or other enclosure made from a water or vapor permeable membrane. Water vapor extraction element 160 may also comprise multiple tubes in parallel, i.e. a “boiler” tube configuration. Exemplary of water permeable membranes are NAFION (Tradename of E. I. Dupont de Nemours & Co., Inc.), a perfluorosulfonate ionomer, polytetrafluoroethylene, fluorinated ethylenepropylene and the like.

Water vapor extraction element 160 is surrounded by a gas tight housing 30. Housing 30 communicates with a vacuum supply 150, via a fourth line 140, and a sump 90. Housing 30 is cooled by thermo-electric cooler 20. Cooler 20 is significantly smaller than cooler 20 (FIG. 1) used in the prior art condensation prevention system 2 because housing 30 is not directly warmed by the flow of shuttle gas. In contrast, note that in FIG. 1, cold trap 30 is directly warmed by the flow of shuttle gas. Consequently, prior art cooler 20 (FIG. 1) consumes 5 Watts as compared to 0.2 Watts consumed by improved condensation system cooler 20.

As the shuttle gas shuttles back and forth between the pump 50 and IAB 10, in catheter 100, the shuttle gas passes through water vapor extraction element 160. During these transits, water vapor in the shuttle gas is transported by water vapor extraction element 160 to an interior space 161 defined by housing 30. The water vapor is removed from interior space 161 by vacuum supply 150, which maintains a water vapor pressure within the housing 30 which is lower than the water vapor pressure in catheter 100. This differential in water vapor pressures is necessary to maintain the diffusion of the water vapor from the shuttle gas through water vapor extraction element 160 into interior space 161. Also, it is advantageous for the level of vacuum within housing 30 to be kept lower than the pressure within water vapor extraction element 160, in order to prevent potential collapse or fatigue of water vapor extraction element 160. Most IABP systems use a compressor and aspirator as part of their pneumatic drive. Thus, the vacuum supply 150 is readily available.

Cooler 20 is used to chill water vapor extraction element 160 in order to increase its water removing efficiency. As a consequence of the cooler 20's action, condensate may form on interior walls of housing 30. This condensate collects in sump 90 and is removed by vacuum source 150. Note, that use of improved condensation prevention system 3 without cooler 20, in all embodiments, is anticipated.

Optionally, it may be appropriate to introduce a small hole in the housing 30's wall. The small hole will cause air to flow into housing 30 through sump 90 and out through vacuum supply 150. This flow will continuously flush interior space 161 of housing 30 with fresh (purge) gas while removing condensate from sump 90. The hole must be small to assure that the pressure within housing 30 remains low.

In an alternate embodiment, the function of vacuum 150 may be replaced with a dry gas flush. A gas, having a lower water vapor content than the shuttle gas, may be used to flush water vapor extraction element 160. Said flush gas removes the water vapor from water vapor extraction element 160. The flush gas may be supplied by a compressor/aspirator (not separately shown, but may be part of pump 50).

It should be noted that exposure of water vapor extraction element 160 to atmosphere without use of either a housing, cooling, and vacuum is anticipated. However, it is believed that use of these elements increases the water vapor removal efficiency of the system.

Note also that sump 90 is optional. If the vacuum created by vacuum supply 150 is kept high enough, then condensate will not appear in housing 30. Specifically, the vacuum level must be sufficient to keep the dew point of the water vapor within housing 30 to a value below ambient temperature.

In the event of an IAB perforation, i.e. a balloon 10 perforation, water vapor extraction element 160 may be contaminated with blood. Due to its simple configuration, this component can be replaced as an assembly.

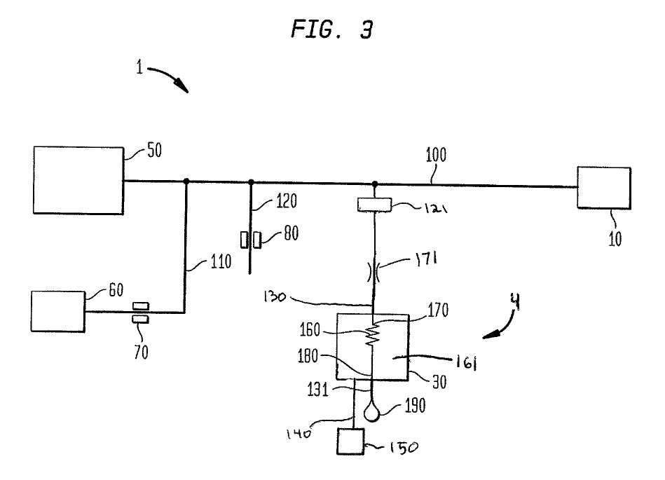

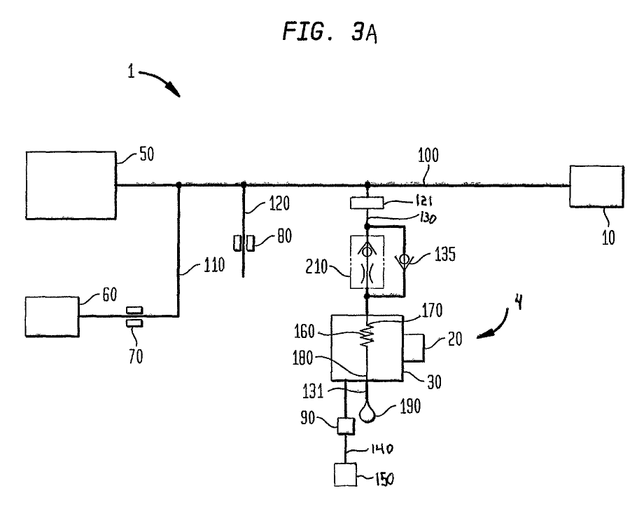

FIG. 3 illustrates a block diagram of the preferred embodiment of the IABP system 1 with an improved condensation prevention system 4 in shunt with catheter 100. In this embodiment, water vapor extraction element 160 is surrounded by a gas tight housing 30. Housing 30 defines an interior space 161 which communicates with a vacuum supply 150, via a fourth line 140. Most IABP systems use a compressor and aspirator as part of their pneumatic drive. Thus, vacuum supply 150 is readily available.

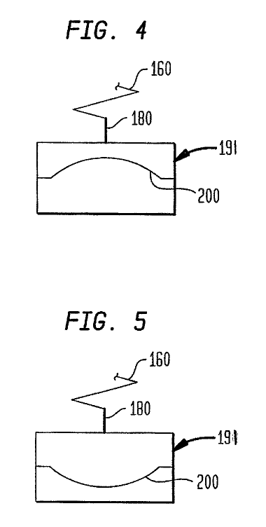

Water vapor extraction element 160 has a first end 170, which is connected to a third line 130, and a second end 180 which is connected to a tidal balloon 190, or alternatively to a safety disk 191 (see FIGS. 4 and 5). The third line 130 connects water vapor extraction element 160 in shunt to catheter 100. A filter 121, connected to third line 130, filters all flow entering condensation prevention system 4. A restriction element 171, also connected to third line 130, restricts flow into and out of condensation prevention system 4. Restriction element 171 allows for control of the dwell time of the shuttle gas in water vapor extraction element 160, the benefits of which are detailed below.

Water vapor extraction element 160 may comprise a tube or any type of enclosure made from a water or vapor permeable membrane. Exemplary of water permeable membranes are, e.g., NAFION (Tradename of E. I. Dupont de Nemours & Co., Inc.), a perfluorosulfonate ionomer, polytetrafluoroethylene, fluorinated ethylenepropylene and the like.

It is preferred that the water vapor extraction element 160 comprise a NAFION tube having a minimum wall thickness of approximately 0.010 inches, an inner diameter between approximately 0.077 inches and 0.094 inches, and an outer diameter between approximately 0.097 inches and 0.119 inches. It is also preferred that water vapor extraction element 160 have a number of bends to maximize the cooling effect of cooler 20.

Improved condensate prevention system 4 operates in the following manner. During normal operation of IABP system 1, the pressure at the junction between catheter 100 and third line 130 rises and falls as a consequence of the pumping action of pump 50. In response to these pressure swings, a portion of the shuttle gas flows back and forth through improved condensate removal system 4 inflating and deflating tidal balloon 190.

During these transits, water vapor in the shuttle gas is transported by water vapor extraction element 160 to an interior space 161 defined by housing 30. The water vapor is removed from interior space 161 by vacuum supply 150, which maintains a water vapor pressure within housing 30 which is lower than the water vapor pressure in catheter 100. This differential in water vapor pressures is necessary to maintain the diffusion of the water vapor from the shuttle gas through water vapor extraction element 160 into interior space 161.

Also it is advantageous if the level of vacuum within the housing 30 is kept lower than the pressure within water vapor extraction element 160, to prevent potential collapse or fatigue of water vapor extraction element 160.

Restrictor 171 slows the inflation and deflation of tidal balloon 190. The restriction is designed to increase the “dwell” time of shuttle gas, i.e. to increase the amount of time the shuttle gas spends in water vapor extraction element 160. This improves the efficacy of water vapor extraction element 160.

To induce the necessary flow of shuttle gas through water vapor extraction element 160, tidal balloon 190 is preferably sized to be a multiple of the volume of water vapor extraction element 160. In the event that the deflation of tidal balloon 190 is not assured by the pressure differential across its walls, it may be biased to deflate either due to its own elasticity or due to the action of a spring. Alternatively, tidal balloon 190 may be biased by placing it in a vented chamber which generates back pressure when tidal balloon 190 inflates. The induced back pressure aids deflation of tidal balloon 190.

A spring loaded rolling diaphragm air cylinder may be used instead of tidal balloon 190 or safety disk 191 (FIGS 4 and 5). In this case, the spring biases the diaphragm into its deflated (empty) position. If safety disk 191 is used, it moves from a low volume position, as illustrated in FIG. 4, to a high volume position, as illustrated in FIG. 5.

There are numerous benefits to connecting condensation prevention system 4 in shunt to catheter 100, a major feature of the present invention. In the shunt configuration only a small portion of the shuttle gas is shunted through condensation prevention system 4 during each inflate/deflate cycle of pump 50. Because the volume of flow through condensation prevention system 4 is much lower in the shunt configuration, as compared to the in-line configuration (FIGS. 1 and 2), the size of the fittings and tubing can be significantly reduced without compromising IAB pumping speed or condensation prevention system 4 operation. Reduction of fitting and tubing size significantly reduces the dead volume associated with the condensate removal function.

In general, a reduction of shuttle gas system dead volume results in a reduction of pump (50) energy consumption and improved shuttle gas leak detection sensitivity. Reduction of dead volume also results in a reduction of the intra-aortic balloon system's pneumatic compliance, which in turn improves therapy. Furthermore, use of the shunt configuration allows for the insertion of filter 121 in series with condensation prevention system 4. Filter 121 is useful to prevent contamination of condensation prevention system 4 components during blood back events, i.e. leakage of blood into IAB balloon 10 through catheter 100 and into condensation prevention system 4.

Filter 121 cannot be placed in-line with prior art condensation prevention system 2 (FIG. 1) or condensation prevention system 3 (FIG. 2) because the pressure drop created by filter 121 would be unacceptably high. Gas flow through an in-line condensation prevention is much higher than through the in-shunt condensation prevention system 4 (FIG. 3). Therefore, the pressure drop associated with insertion of an in-line filter would be proportionally higher.

FIGS. 4 and 5 illustrate a second end 180 of water vapor extraction element 160 terminating in safety disk 191. Safety disk 191 comprises a membrane 200 which moves back and forth from the position illustrated in FIG. 4 to the position illustrated in FIG. 5 in response to changes in shuttle gas pressure in water vapor extraction element 160. Movement of membrane 200 (FIGS. 4 and 5) allows the shuttle gas to flow from catheter 100 into water vapor extraction element 160.

FIG. 3A illustrates the condensation prevention system 4 of FIG. 3 with some optional additional features. Cooler 20 is used to cool housing 30. Vacuum supply 150 is connected to housing 30 via line 140. A sump 90, for collection of accumulated condensate, is connected to line 140 between housing 30 and vacuum supply 150. As discussed previously, these components may be added to increase the efficacy of condensate removal system 4.

Also illustrated in FIG. 3A is a restricted check valve 210, connected on third line 130, and second check valve 135, connected in parallel with restricted check valve 210 and acting as a one-way bypass of restricted check valve 210. Restricted check valve 210 allows for flow of shuttle gas into water vapor extraction element 160 disposed within housing 30. Check valve 135 allows for flow of shuttle gas out of tidal balloon 190 through water vapor extraction element 160 and through check valve 135. The function of these components is discussed below.

In IABP systems, loss of shuttle gas is detected by measuring the pressure of the shuttle gas when the patient balloon (IAB) is in its fully deflated state. At this time, the total volume of the shuttle gas system is known and fixed, therefore, a drop in shuttle gas pressure corresponds to a loss of shuttle gas. To be valid pressure reading, the shuttle gas must be in a static state, i.e. there cannot be any flow. Hence, the pressure reading is taken just prior to IAB inflation, this method allows for the longest time to deflate the IAB, thereby assuring that gas flow from the IAB has ceased.

In the case of the condensate removal system's tidal balloon 190, there is a similar need to assure its full deflation prior to measurement of shuttle gas pressure for leak detection. FIG. 3A, discloses a check valve/restrictor arrangement which causes tidal balloon 90 to inflate and deflate in an asymmetric fashion. Specifically, check valve 135 assures that the tidal balloon deflates rapidly, which is desirable for the reasons stated above, and check valve 210 and restrictor 210 assure that the inflation of tidal balloon 190 is slowed. The efficacy of water vapor extraction element 160 is improved if the flow through it is slowed.

According to “Efficiency and Temperature Dependence of Water Removal by Membrane Dryers,” by Leckrone, et. al. Analytical Chemistry Vol. 69, Number 5, pages 911-918, the efficiency of Nafion's vapor removal process can be optimized by using a low flow rate through the water vapor extraction element 160. This is a consequence of the finite amount of time required to transport water through Nafion tubing. Accordingly, the invention optionally includes restricted check valve 210 (FIG. 3) or restriction 171 (FIG. 2) on the third line 130. The restriction is designed to increase the “dwell” time of shuttle gas, i.e. to increase the amount of time the shuttle gas spends in water vapor extraction element 160. The theory is that the slower the shuttle gas is traveling through water vapor extraction element 160 the more time there is for it to dry. Restricted check valve 210 assures that the drying capacity of water extraction element 160 is fully utilized.

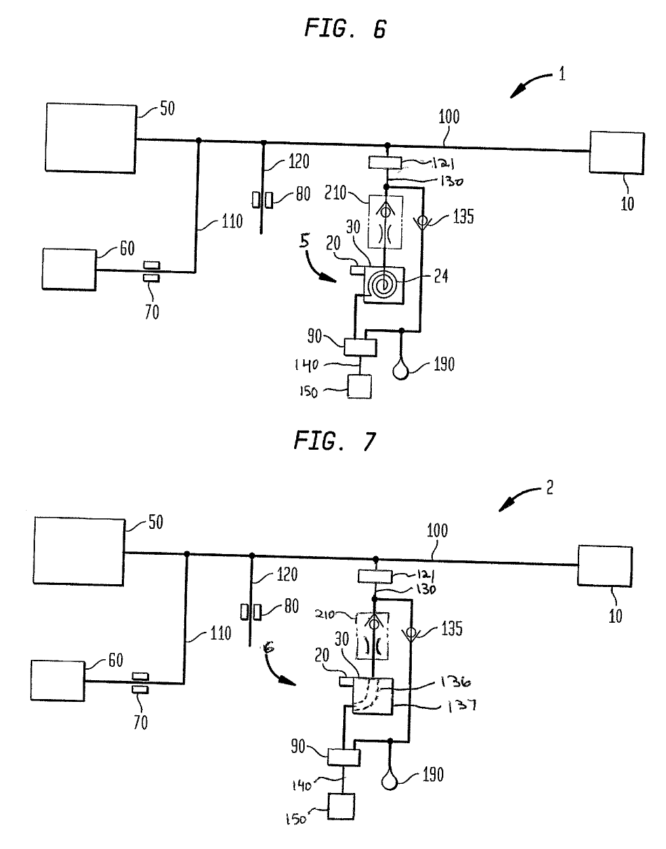

FIG. 6 illustrates a block diagram of another embodiment of the improved IABP system 1 having an improved condensate prevention system 5 comprising a filter 121, a sump 90, a vacuum supply 150 connected to the sump 90, a water vapor condensing element 24, a housing 30 located between the filter 121 and the sump 90 and containing water vapor condensing element 24, a cooler 20 for cooling housing 30, a tidal balloon 190, a restricted check valve 210, and a check valve 135.

This embodiment is different in that (a) water vapor condensing element 24 replaces water vapor extraction element 160 as utilized in the embodiment illustrated in FIGS. 2, 3, and 3A and (b) housing 30 may be filled with a fluid, or gel, or other appropriate conductive filler to facilitate heat transfer between water vapor condensing element 24 and cooled housing 30.

Water vapor condensing element 24, unlike water vapor extraction element 160 (FIGS. 1, 2, 3, and 3A), is not made from a water vapor permeable membrane. Rather water vapor condensing element 24 may be made from conventional tubing materials such as metal or plastic, and is designed to induce condensation within its walls and to route this condensation to sump 90.

Note that it is anticipated that the water vapor condensing element 24 may be replaced with a section or enclosure having another geometry, including a straight or V-shaped tube as used in the prior art, see FIG. 1.

A coil is preferred, however, because it exposes more tube surface area to the lower temperature created by cooler 210. Possible alternative elements discussed for the other embodiments are equally applicable for this and all other embodiments of the invention.

As shuttle gas is shuttled from pump 50 to IAB 10 some shuttle gas flows into third line 130, passes through restricted check valve 210 and into water vapor condensing element 24. The cooled coiled water vapor condensing element 24 behaves as a water vapor condenser and causes at least some of the water vapor in the shuttle gas shuttle gas to condense. Water droplets are forced towards sump 90, connected between vacuum supply 150 and cold trap 30, by the intermittent flow of shuttle gas form catheter 100. An inner surface of the water vapor condensing element 24 may be coated with a hydrophobic coating so as to facilitate the migration of condensed water droplets to sump 90.

As shuttle gas is shuttled back from IAB 10 to pump 50, tidal balloon 190 deflates and empties through check valve 135. The arrangement of check valve 135 and restricted check valve 210 assures that the flow through water vapor condensing element 24 unidirectional. This valve system assures a more steady and efficient migration of condensed water droplets in water vapor condensing element 24 in to sump 90's inlet. Note that tidal balloon 190 is placed on the outlet side of sump 90. This keeps liquid water from entering tidal balloon 190.

Note that use of condensate prevention system 5 without restricted check valve 210, check valve 135, or any other restrictive element to assure steady migration of condensed water droplets to sump 90, is anticipated. FIG. 7 illustrates an alternate embodiment of the present invention, similar to the embodiment illustrated in FIG. 6 except for the use of the block condensate prevention system 6. Block condensate prevention system 6 comprises a block 137 having a drilling 136 through it. Block 137 is similar to cold trap 30 used in the prior art (FIG. 1) except for its placement in shunt with catheter 100. Tube 130 is connected to an inlet of block 137 and tube 140 is connected to an outlet of block 137. Both tube 137 and tube 140 communicate with drilling 136. Note that drilling 136 may take on other shapes, including a coil shape, to increase the cooling effect of cooler 20. Similar to the embodiment in FIG. 6, the optional arrangement of check valve 135 and restricted check valve 210 assures that the flow through drilling 136 is unidirectional.

Note, as in the embodiment illustrated in FIGS. 3 and 3A, safety disk 191 or a spring loaded rolling diaphragm air cylinder may be used instead of tidal balloon 190. Note also that the use of tidal balloon 190, and its alternatives, in FIGS. 6 and 7, are not absolutely necessary as sump 90 itself acts as a tidal volume. However, the use of a tidal volume having flexible walls is preferred.

What is claimed is:

1. A method for operating an intra-aortic balloon pump system comprising a balloon pump, an intra-aortic balloon catheter connected to said pump and terminating in a balloon membrane, a condensate prevention system, and a valve system connected between the condensate prevention system and the intra-aortic balloon catheter,
said condensate prevention system comprising a water vapor extraction element communicating with the intra-aortic balloon catheter, said intra-aortic balloon pump containing a predetermined amount of shuttle gas for inflation and deflation of the balloon membrane,
comprising the steps of:
(a) pumping a predetermined amount of a shuttle gas through the intra-aortic balloon catheter;
(b) opening the valve system so as to allow the shuttle gas to pass into water vapor extraction element;
(c) inflating the balloon membrane;
(d) pumping said predetermined amount of shuttle gas in the opposite direction as in step (a);
(c) at least partially closing the valve system for a predetermined amount of time so as to trap a predetermined amount of shuttle gas in the water vapor extraction element;
(d) deflating the balloon membrane;
(e) repeating steps (a) through (d).

2. The method as claimed in claim 1 wherein the condensate prevention system further comprises an expansion means connected to one end of the water vapor extraction element and wherein the valve system is kept at least partially open for a predetermined amount of time sufficient to allow the shuttle gas to enter both the water vapor extraction element and the expansion means.

3. The method as claimed in claim 1 wherein the valve system comprises a check valve and a restricted check valve in shunt connected between the water vapor extraction element and the intra-aortic balloon catheter.

4. The method as claimed in claim 1 wherein the condensate prevention system further comprises a housing surrounding the water vapor extraction element communicating with a vacuum supply and further comprising the step of applying the vacuum to the housing so as to reduce the pressure in said housing.

5. The method as claimed in claim 1 wherein the condensate prevention system further comprises a housing surrounding the water vapor extraction element and further comprising the step of purging an interior of said housing with a purge gas which is drier than the shuttle gas.

6. The method as claimed in claims 4 or 5 further comprising the step of cooling the housing.

7. The method as claimed in claim 2 wherein the expansion means comprises a balloon.

8. The method as claimed in claim 2 wherein the expansion means comprises a safety disk.

9. A method for operating an intra-aortic balloon pump system comprising a balloon pump, an intra-aortic balloon catheter connected to said pump and terminating in a balloon membrane, a condensate prevention system, and a valve system connected between the water vapor removal system and the intra-aortic balloon catheter,
said condensate prevention system comprising a water vapor extraction element communicating with the intra-aortic balloon catheter, said intra-aortic balloon pump containing a predetermined amount of shuttle gas for inflation and deflation of the balloon membrane,
comprising the steps of:
(a) at least partially opening the valve system so as to allow the shuttle gas to pass into water vapor extraction element;
(b) inflating the balloon membrane by pumping a predetermined amount of a shuttle gas through the intra-aortic balloon catheter;
(c) at least partially closing the valve system for a predetermined amount of time so as to trap a predetermined amount of shuttle gas in the water vapor extraction element;
(d) deflating the balloon membrane by pumping said predetermined amount of shuttle gas in the opposite direction as in step (a); and
(e) repeating steps (a) through (d).

10. A method for operating an intra-aortic balloon pump system comprising a balloon pump, an intra-aortic balloon catheter connected to said pump and terminating in a balloon membrane, a condensate prevention system, and a valve system connected between the condensate prevention system and the intra-aortic balloon catheter,
said condensate prevention system comprising a water vapor extraction element communicating with the intra-aortic balloon catheter, said intra-aortic balloon pump containing a predetermined amount of shuttle gas for inflation and deflation of the balloon membrane,
said valve system comprising a first one-way valve and a second one-way valve in shunt connected between the water vapor extraction element and the intra-aortic balloon catheter,
comprising the steps of:
(a) opening the first one-way valve and inflating the balloon membrane by pumping a predetermined amount of a shuttle gas through the intra-aortic balloon catheter;
(b) closing the first one way valve, opening at least partially the second one-way valve, and deflating the balloon membrane by pumping said predetermined amount of shuttle gas in the opposite direction as in step (a); and
(c) repeating steps (a) through (b).

11. A method for operating an intra-aortic balloon pump system comprising a balloon pump, an intra-aortic balloon catheter connected to said pump and terminating in a balloon membrane, a condensate prevention system, and a restriction system connected between the water vapor removal system and the intra-aortic balloon catheter,
said condensate prevention system comprising a water vapor extraction element communicating with the intra-aortic balloon catheter, said intra-aortic balloon pump containing a predetermined amount of shuttle gas for inflation and deflation of the balloon membrane,
comprising the steps of:
(a) allowing the shuttle gas to pass the restriction system into the water vapor extraction element;
(b) inflating the balloon membrane by pumping a predetermined amount of a shuttle gas through the intra-aortic balloon catheter;
(c) trapping for a predetermined amount of time a predetermined amount of shuttle gas in the water vapor extraction element;
(d) deflating the balloon membrane by pumping said predetermined amount of shuttle gas in the opposite direction as in step (a); and
(e) repeating steps (a) through (d).

12. The method as claimed in claims 1, 2, 3, 4, 5, 7, 8, 9, 10, or 11 wherein the water vapor extraction element comprises a tube made from a water vapor permeable membrane.

13. The method as claimed in claims 1, 2, 3, 4, 5, 7, 8, 9, 10, or 11 wherein the water vapor extraction element comprises a tube made from a perfluorosulfonate ionomer.

14. The method as claimed in claims 1, 9, 10, or 11 wherein the condensation prevention system is connected in shunt to the intra-aortic balloon catheter.

15. An intra-aortic balloon pump system comprising a pump, an intra-aortic balloon catheter, and a water vapor extraction element connected in shunt to the intra-aortic balloon catheter, said intra-aortic balloon catheter comprising a catheter having a proximal and distal end, said catheter proximal end being connected to the pump and said catheter distal end terminating in a balloon membrane.

16. The intra-aortic balloon pump system as claimed in claim 15 wherein the water vapor extraction element comprises a tube made from a water vapor permeable membrane.

17. The intra-aortic balloon pump system as claimed in claim 15 wherein the water vapor extraction element comprises a tube made from a perfluorosulfonate ionomer.

18. The intra-aortic balloon pump system as claimed in claim 15 wherein the water vapor extraction element comprises a tube made from a water vapor permeable membrane and wherein one end of the tube is connected in shunt to the intra-aortic balloon catheter and the opposite end of the tube is connected to an expansion means.

19. The intra-aortic balloon pump system as claimed in claim 15 wherein the water vapor extraction element comprises a tube made from a water vapor permeable membrane and wherein one end of the tube is connected in shunt to the intra-aortic balloon catheter and the opposite end of the tube is connected to a balloon.

20. The intra-aortic balloon pump system as claimed in claim 15 wherein the water vapor extraction element is disposed in a housing.

21. The intra-aortic balloon pump system as claimed in claim 15 wherein the water vapor extraction element is disposed in a housing, said housing being connected to a vacuum means.

22. The intra-aortic balloon pump system as claimed in claim 15 wherein the water vapor extraction element is disposed in a housing connected to a purge means for purging the water vapor extraction element with a purge gas.

23. The intra-aortic balloon pump system as claimed in claim 15 further comprising a restrictive system connected between the intra-aortic balloon catheter and the water vapor extraction element.

24. The intra-aortic balloon pump system as claimed in claim 15 further comprising a restrictive system connected between the intra-aortic balloon catheter and the water vapor extraction element, said restrictive system comprising a restricted check valve and a check valve connected in parallel between the water vapor extraction element and the intra-aortic balloon catheter.

25. The intra-aortic balloon pump system as claimed in claim 15 further comprising a filter connected between the water vapor extraction element and the intra-aortic balloon catheter.

26. An intra-aortic balloon pump system comprising a pump, an intra-aortic balloon catheter, and a water vapor condensing element connected in shunt to the intra-aortic balloon catheter, said intra-aortic balloon catheter comprising a catheter having a proximal and distal end, said catheter proximal end being connected to the pump and said catheter distal end terminating in a balloon membrane.

27. The intra-aortic balloon pump system as claimed in claim 26 wherein the water vapor condensing element comprises a tube.

28. The intra-aortic balloon pump system as claimed in claim 26 wherein the water vapor condensing element comprises a coiled tube and wherein one end of the tube is connected in shunt to the intra-aortic balloon catheter and the opposite end of the tube is connected to an expansion means.

29. The intra-aortic balloon pump system as claimed claim 26 wherein the water vapor condensing element comprises a coiled tube and wherein one end of the tube is connected in shunt to the intra-aortic balloon catheter and the opposite end of the tube is connected to a balloon.

30. The intra-aortic balloon pump system as claimed in claim 26 wherein the water vapor condensing element is disposed in a housing.

31. The intra-aortic balloon pump system as claimed in claim 26 wherein the water vapor condensing element is disposed in a housing at least partially filled with a thermally conductive filling substance.

32. An intra-aortic balloon pump system comprising a pump, an intra-aortic balloon catheter, and a cold trap connected in shunt to the intra-aortic balloon catheter, said intra-aortic balloon catheter comprising a catheter having a proximal and distal end, said catheter proximal end being connected to the pump and said catheter distal end terminating in a balloon membrane, said cold trap comprising a block having a drilling in it, said drilling defining a lumen, said lumen communicating with the intra-aortic balloon catheter.

33. The intra-aortic balloon pump system as claimed in claim 32 further comprising a filter and a restrictor connected between the intra-aortic balloon catheter and the cold trap.
